(19) United States
(12) Patent Application Publication
(10) Pub. No.: US 2011/0091957 A1
(43) Pub. Date: Apr. 21, 2011

(54) PROTEIN-POLYMER CONJUGATES AND SYNTHESIS THEREOF

(76) Inventors: BHALCHANDRA SHRIPAD LELE, PITTSBURGH, PA (US); ALAN J. RUSSELL, GIBSONIA, PA (US)

(21) Appl. No.: 12/965,024

(22) Filed: Dec. 10, 2010

Related U.S. Application Data
(63) Continuation of application No. 11/520,182, filed on Sep. 13, 2006.
(60) Provisional application No. 60/716,456, filed on Sep. 13, 2005.

(51) Int. Cl. C07K 1/00; C12N 9/96

(52) U.S. Cl. 435/188; 527/201; 527/202; 530/402

(57) ABSTRACT

A method of synthesizing a protein-polymer conjugate includes the steps: covalently attaching at least one controlled radical polymerization initiator to a protein to form a protein-initiator composition; and mixing the protein-initiator composition with at least one monomer which undergoes controlled radical polymerization in the presence of the protein-initiator composition under conditions suitable to initiate the controlled radical polymerization.

### CROSS-REFERENCE TO RELATED APPLICATION

This application is a continuation of U.S. patent application Ser. No. 11/520,182, filed Sep. 13, 2006, which claims benefit of U.S. Provisional Patent Application No. 60/716,456 filed Sep. 13, 2005, the disclosure of which is incorporated herein by reference.

### GOVERNMENT INTEREST

This invention was made with government support under grant number DAAD: 19-01-0619 from the Department of Defense. The government has certain rights in this invention.

### BACKGROUND OF THE INVENTION

The present invention relates protein-polymer conjugates and synthesis thereof and, particularly, to protein-polymer conjugates synthesized via a controlled radical polymerization.

The following information is provided to assist the reader to understand the invention disclosed below and the environment in which it will typically be used. The terms used herein are not intended to be limited to any particular narrow interpretation unless clearly stated otherwise in this document. References set forth herein may facilitate understanding of the present invention or the background of the present invention. The disclosure of all references cited herein are incorporated by reference.

Biopharmaceuticals increasingly rely on uniform protein-polymer conjugates for consistent and sustainable therapeutic effects. Grace, M. J. et al. J. Biol. Chem. 2005, 280, 6327-6336; Rosendahl, M. S.; Doherty, D. H.; Smith, D. J.; Clarson, S. J.; Chlipala, E. A.; Cox, G. N. Bioconjugate Chem. 2005, 16, 200-207. Uniformity in protein modification is achievable either by site-specific polymer attachment to unique thiol/amine groups, genetically introduced in proteins or by de novo protein synthesis from polymer-attached amino acids. See, for example, Tsutsumi, Y.; Onda, M.; Nagata, S.; Lee, B.; Kreitman, R. J.; Pastan, I. Proc. Natl. Acad. Sci. USA 2000, 97, 8548-8553; Yamamoto, Y. et al. Nature Biotechnol. 2003, 21, 546-552; and Kochendoerfer, G. G. et al. Science 2003, 299, 884-887. These approaches are high cost, low yield and not generic. Because of the plurality of lysine residues in native proteins that result in variable degrees of modification in simple conjugations there has been no straightforward way to perform uniform protein modifications with polymers.

Conventionally, monomethoxy poly(ethylene glycol) (MPEG) bearing an activated carboxyl end group has been used to modify proteins and increase their stability. See, for example, Harris, J. M.; Chess, R. B. Nature Rev. Drug Disc. 2003, 2, 214-221. Kynclova et al used an alternative approach for protein PEGylation by activating the carboxyl groups of lipase and conjugating O,O-bis-(2-aminopropyl)polyethylene glycol to the protein. See Kynclova, E.; Elsner, E.; Köpf, A.; Hawa, G.; Schalkhammer, T.; Pittner, F. J. Mol. Recognit. 1996, 9, 644-651. This approach resulted in the formation of PEGylated lipase in which the number of conjugated PEG chains per protein molecule was 2.5 to 4. Recently, combed shaped and reactive-end-group-bearing poly(MPEG-methacrylate)s with low polydispersity indices (synthesized by ATRP) have been used to modify proteins. See, for example, Tao, L.; Mantovani, G.; Lecolley, F.; Haddleton, D. M. J. Am. Chem. Soc. 2004, 126, 13220-13221 and Mantovani, G.; Lecolley, F.; Tao, L.; Haddleton, D. M.; Clerx, J.; Cornelissen, J. J. L. M.; Velonia, K. J. Am. Chem. Soc. 2005, 127, 2966-2973. The branched structure of the polymer can improve the conjugates' resistance to proteolysis and to the action of antibodies resulting in lower immunogenicity. See, for example, Veronese, F. M.; Monfardini, C.; Caliceti, P.; Schiavon, O.; Scrawen, M. D.; Beer, D. J. Controlled Rel. 1996, 40, 199-209 and Schiavon, O.; Caliceti, P.; Ferruti, P.; Veronese, F. M. Il Pharmaco 2000, 55, 264-269.

Use of proteins as ATRP initiators would allow control over both the number of poly(MPEG-methacrylate) chains attached to a protein and the polydispersity of the growing chain. A recent study by Bontempo and Maynard used streptavidin-biotinylated ATRP initiator complex in conjunction with a sacrificial initiator to synthesize a streptavidin-biotin-poly(N-isopropylacrylamide) conjugate. Bontempo, D.; Maynard, H. D. J. Am. Chem. Soc. 2005, 127, 6508-6509. Streptavidin is known to noncovalently bind biotin. In the study of Bontempo, a known biotinylated ATRP initiator was bound to streptavidin via the biotin ligand to form a protein-biotin initiator complex. The protein-biotin-initiator complex alone could not, however, initiate the polymerization. The bulk of the initiator, a relatively poor initiation efficiency and a low concentration of initiators in the studies of Bontempo and Maynard may have prevented initiation of polymerization without a sacrificial initiator. Furthermore, the reaction conditions used in the reactions of those studies were not suitable to maintain biological activity of the protein.

Klok recently reviewed polymer-oligopeptide block copolymer synthesis by peptide-initiated ATRP and speculated about the emerging possibility of precisely controlling the growth of polymers from protein surfaces. Klok, H-A. J. Polym. Sci. Polym. Chem. 2005, 43, 1-17. There are a number of predictable barriers that might prevent the growth of uniform polymer chains from proteins. These barriers include uncontrollable modification of lysines with initiators, cross-linking of growing chains and most importantly, the loss in activity of the protein either during or after polymerization.

It remains desirable to develop protein-polymer conjugates and improved methods for synthesis thereof.

### SUMMARY OF THE INVENTION

In one aspect, the present invention provides a method of synthesizing a protein-polymer conjugate including the steps: covalently attaching a controlled radical polymerization initiator to a protein to form a protein-initiator composition; and mixing the protein-initiator composition with at least one monomer which undergoes controlled radical polymerization in the presence of the protein-initiator composition under conditions suitable to initiate the controlled radical polymerization.

The controlled radical polymerization can, for example, be atom transfer radical polymerization. The atom transfer radical polymerization preferably occurs under conditions suitable to maintain biological activity in the protein-polymer conjugate. The atom transfer radical polymerization can, for example, occur under conditions suitable to maintain a biological activity in the protein-polymer conjugate of at least 10%, 25%, 50% or even 75% as compared to a natural biological activity of the protein. In general, maintained or retained biological activity is preferably maximized.

The atom transfer radical polymerization can, for example, occur in an aqueous environment. The atom transfer radical polymerization can also occur in the presence of a non-aqueous solvent. For example, an ion pair of a surfactant and the initiator-protein composition can be dissolved in the non-aqueous solvent and polymerization and atom transfer radical polymerization is then initiated.

In several embodiments, the atom transfer radical polymerization occurs at a temperature in the range of 0° C. to 90° C. The atom transfer radical polymerization can also occur at a temperature of less than 50° C. or a temperature of less than 30° C. In several embodiments, the atom transfer radical polymerization occurs at a temperature of between 0° C. to 25° C.

The protein-initiator can, for example, have only one atom transfer radical polymerization initiator covalently attached thereto. The protein-initiator can also have more than one atom transfer radical polymerization initiator covalently attached thereto. The number of initiators covalently attached to the protein can, for example, be controlled by control of the molar ratio of initiator to protein. The molar ratio can, for example, controlled to achieve a single initiator covalently attached to the protein, two initiators covalently attached to the protein or more initiators covalently attached to the protein. The ratio of monomer concentration to initiator group concentration can, for example, be 1 to 100,000.

The step of covalently attaching a controlled radical polymerization initiator to a protein to form a protein-initiator can, for example, be an acylating or alkylating reaction. The pH is of the reaction can, for example, be in the range of approximately 4 to 11.

The monomer(s) used in the present invention can, for example, include at least one of mPEG-methacrylate, vinyl pyrrolidone, 2-hydroxypropyl methacrylamide, N-isopropylacrylamide, O-acryloyl-N-acetyl glucosamine, 2-hydroxyethyl methacrylate, dimethylaminoethyl methacrylate, styrene sulfonic acid, N-acryloxysuccinimide, acrylic acid, methacrylic acid, styrene, methyl methacrylate, butyl methacrylate, 2-ethyl hexyl methacrylate, poly(propylene glycol)methacrylate, perfluoroalkyl methacrylate, vinyl acetate, lauryl acrylate or various derivatives of the listed monomers. As clear to one skilled in the art, many other monomers suitable for radical polymerization in aqueous or organic solvent environments are suitable for use in the present invention. The initiator can, for example, include, at least one of 2-bromoisobutyryl bromide, 2-bromo-2-methyl propionic acid, a maleimide derivative of 2-bromo-2-methyl propionic acid, 2-chloro-2-methyl-propionic acid, a chloro phenyl carboxylic acid, a bromomethyl phenyl carboxylic acid or various derivatives of the listed initiators. As clear to one skilled in the art, many other initiators are suitable for use in the present invention. As also clear to one skilled in the art, virtually any protein is suitable for use in the present invention. Examples of such proteins include, but are not limited to, chymotrypsin, subtilisin, lipase, peroxidase, chloroperoxidase, epoxidase, asparaginase, interferon, a tumor necrosis factor, insulin, superoxide dismutase, or a growth factor. The protein can, for example, be a biocatalyst or a pharmaceutical agent.

In another aspect, the present invention provides a composition including a protein having covalently attached thereto a controlled radical polymerization initiator. The initiator can, for example, be an atom transfer radical polymerization initiator.

In still a further aspect, the present invention provides a composition including a protein having covalently attached thereto a linking moiety. The linking moiety has covalently attached thereto a polymer chain produced by a controlled radical polymerization. The linking moiety corresponds to an initiator covalently attached to the protein that was used in initiating the controlled radical polymerization. The initiator can, for example, be an atom transfer radical polymerization initiator.

In general, the present invention provides for modification a reactive group on a protein with an initiator and then growing a polymeric chain from that site to, for example, achieve substantial uniformity in terms of the number and length of polymer chains per protein. We describe a technique to synthesize enzyme-polymer conjugates containing near-monodisperse polymer chains, the functionality of which can be modified through straightforward chemistry. We achieve this significant step toward synthesizing biologically active polymer-protein machines by growing the polymer chain via a controlled radical polymerization such as atom transfer radical polymerization (ATRP) from an initiator molecule conjugated to the model protein.

The present invention, along with the attributes and attendant advantages thereof, will best be appreciated and understood in view of the following detailed description taken in conjunction with the accompanying drawings.

### BRIEF DESCRIPTION OF THE DRAWINGS

FIG. 1 illustrates a schematic representation of a method of the present invention to synthesize uniform protein-polymer conjugate by growing polymer chain from protein.

FIG. 2 illustrates MALDI-TOF spectra of native chymotrypsin and 2-bromoisobutyramide derivatives of chymotrypsin (protein-initiators CT¹*, CT⁴*, CT⁸*).

FIG. 3(a) illustrates an SEC-LS chromatogram of native chymotrypsin. Mw=26,700 Da, PDI=1.05.

FIG. 3(b) illustrates an SEC-LS chromatogram of CT¹*-graft-poly(MEPG-methacrylate). Mw=45,600 Da, PDI=1.05.

FIG. 3(c) illustrates an SEC-LS chromatogram of conventionally synthesized conjugate of chymotrypsin with MPEG-SPA. Mw=32,400 Da, PDI=1.34.

FIG. 3(d) illustrates an MALDI-TOF spectrum of conventionally synthesized conjugate of chymotrypsin with MPEG-SPA.

FIG. 3(e) illustrates an SEC-LS chromatogram of conventionally synthesized conjugate of chymotrypsin with carboxyl-functionalized poly(MPEG-methacrylate). Mw=209,300 Da, PDI=1.66.

FIG. 4 illustrates an SDS-PAGE for: (1) Molecular weight markers. (2) Native chymotrypsin (after incubating at 40° C. for 16 h. Two bands are observed, one for the enzyme at 28 kDa marker level and the other for the impurity at 18 kDa marker level (which is also seen in SEC-LS chromatogram at elution time 10-11 min); and (3) CT¹*-graft-poly(MPEG-methacrylate) (4) CT⁴*-graft-poly(MEPG-methacrylate).

FIG. 5(a) and (b) illustrates a MALDI-TOF spectra of chymotrypsin lysate obtained after trypsin digestion.

FIGS. 5(c) and (d) illustrate a MALDI-TOF spectra of CT¹*-graft-poly(MPEG-methacrylate) lysate obtained after trypsin digestion.

FIG. 6(a) illustrates an SEC-LS chromatogram of native chymotrypsin. Mw=26,700 Da, PDI=1.05.

FIG. 6(b) illustrates an SEC-LS chromatogram of CT⁴*-graft-poly(sodium 4-styrenesulfonate). Mw=78,150 Da, PDI=1.10.

FIG. 6(c) illustrates an SEC-LS chromatogram of CT⁴*-graft-poly(MPEG475-methacrylate). Mw=52,200 Da, PDI=1.08.

FIG. 6(d) illustrates an SEC-LS chromatogram of CT⁴*-graft-poly(DMAEMA). Mw=44,300 Da, PDI=1.35 (for the broad peak with elution time 8-10 minutes).

### DETAILED DESCRIPTION OF THE INVENTION

In one aspect, the present invention provides a method of synthesizing active protein-polymer conjugates by conducting a controlled/living radical polymerization such as atom transfer radical polymerization of monomers initiated from novel protein—initiator compositions. The resulting protein-polymer conjugates can, for example, be nearly monodisperse in terms of the number and molecular weight of polymer chains attached per protein molecule. The methods of the present invention enable the design and synthesis of well defined and functionally wide ranging polymer-protein conjugates useful, for example, in medicine, catalysis and sensing.

Controlled/living radical polymerization process (CRPs) such as atom transfer radical polymerization (ATRP) provide a relatively new and versatile method for the synthesis of polymers with controlled molecular weights and low polydispersities. See, for example, Lin, J., Tiller, J. C., Lee, S. B., Lewis, K. & Klibanov, A. M. (2002) Biotechnol. Lett. 24, 801-805; Wang, J.-S. & Matyjaszewski, K. (1995) J. Am. Chem. Soc. 117, 5614-5615; Matyjaszewski, K. & Xia, J. (2001) Chem. Rev. 101, 2921-2990. ATRP, nitroxide mediated polymerization (NMP), reversible addition fragmentation chain transfer (RAFT) and catalytic chain transfer (CCT) are examples of controlled/living radical polymerization processes or CRP that provide versatile methods for controlled synthesis of polymers. In general, CRP processes provide compositionally homogeneous well-defined polymers (with predictable molecular weights, narrow molecular weight distribution, and high degree of end-functionalization) and have been the subject of a number of recent review articles. See, for example, Chen, R., Worley, S. D., Kim, Wei, C. I., Chen, T. Y., Santiago, J. I., Williams, J. F. and Sun, G., (2003) Ind. Eng. Chem. Res., 42, 280-284 and Tiller, J. C., Liao, C. J., Lewis, K. and Kilbanov, A. M. (2001), Proc. Nat'l Acad. Sci. USA, 98, 5981-5985.

ATRP is one of the most robust CRP and a large number of monomers can be polymerized providing compositionally homogeneous well-defined polymers having predictable molecular weights, narrow molecular weight distribution, and high degree of end-functionalization. For this reason, ATRP was used in a number of representative studies of the present invention.

Matyjaszewski and coworkers have produced a number of patents, patent applications and journal articles related to ATRP. See, for example, U.S. Pat. Nos. 5,763,548; 5,807,937; 5,789,487; 5,945,491; 6,111,022; 6,121,371; 6,124,411; 6,162,882; 6,407,187; 6,512,060; 6,538,091, 6,541,580, 6,624,262, 6,624,263, 6,627,314, 6,790,919 and 6,887,962 and Published U.S. Patent Application No. 2002/0183473; and; Matyjaszewski, K., Ed. Controlled Radical Polymerization; ACS: Washington, D.C, 1998; ACS Symposium Series 685; Matyjaszewski, K., Ed. Controlled/Living Radical Polymerization. Progress in ATRP, NMP, and RAFT; ACS: Washington, D.C, 2000; ACS Symposium Series 768; Matyjaszewski, K., Davis, T. P., Eds. Handbook of Radical Polymerization; Wiley: Hoboken, 2002; Qiu, J.; Charleux, B.; Matyjaszewski, K. Prog. Polym. Sci. 2001, 26, 2083; and Davis, K. A.; Matyjaszewski, K. Adv. Polym. Sci. 2002, 159, 1, the disclosures of which are incorporated herein by reference.

As used herein, “ATRP” or “atom transfer radical polymerization” refer generally to a controlled/living radical polymerization as, for example, described by Matyjaszewski in the Journal of Americal Chemical Society, vol. 117, page 5614 (1995), as well as in ACS Symposium Serves 768, and Handbook of Radical Polymerization, Wiley: Hobolcer 2002, Matyiaszewski, K and Davis, T, editors, all hereby incorporated by reference.

As used herein, the term “protein” refers generally to biopolymers that include amino-acid residues joined by peptide bonds These biopolymers typically have molecular weights of 5,000 or greater. Proteins contain the elements carbon, hydrogen, nitrogen, oxygen, usually sulfur, and occasionally other elements (as phosphorus or iron). As used herein, the term “enzyme” refers generally to proteins that catalyze biochemical reactions.

ATRP uses one or more monomers (radically polymerizable alkenes), an initiator (typically with a transferable halogen), and a catalyst composed of a transition metal with a suitable ligand. An “ATRP initiator” is a chemical molecule, with a transferable (pseudo)halogen that can initiate chain growth. In general, the initiator can be any compound having one or more atom(s) or group(s) which are radically transferable under the polymerizing conditions. Relatively fast initiation is preferred to obtain well-defined polymers with low polydispersities. A variety of initiators, typically alkyl halides, have been used successfully in ATRP. Many different types of halogenated compounds are potential initiators. Reversible atom transfer can occur between the transition metal complex and the growing radicals, thereby reducing the free radical concentration and decreasing the probability of termination by radical coupling. Suitable initiators, transition metal compounds and ligands for use in the present invention are set forth in the references provided above.

In the protein-initiator compositions of the present invention, the initiator is covalently bound, either directly or via a spacer as in the following formula: P—S—I, wherein P is the protein, S is a spacer (which can be present or can be absent) and I is an initiator. In general, a reactive group on the spacer or the initiator is reacted with a corresponding reactive group on the protein (for example, an amino group —NH₂) to covalently bond the initiator to the protein. The covalently bonded initiator retains the ability to initiate polymer chain growth. It was found that the protein-initiator compositions of the present invention initiated polymerization without the use of a sacrificial initiator The initiator and any spacer group preferably provides for initiation of polymer chain growth with retention of significant biological activity of the protein. The molecular weight, size or bulk of any spacer group is preferably maintained relatively low to prevent significant interference with initiation.

Initiators and monomers suitable for use in the present invention are described, for example, in U.S. Pat. Nos. 5,763,548; 5,807,937; 5,789,487; 5,945,491; 6,111,022; 6,121,371; 6,124,411; 6,162,882; 6,407,187; 6,512,060; 6,538,091, 6,541,580, 6,624,262, 6,624,263, 6,627,314, 6,790,919 and 6,887,962 and Published U.S. Patent Application No. 2002/0183473; and; Matyjaszewski, K., Ed. Controlled Radical Polymerization; ACS: Washington, D.C, 1998; ACS Symposium Series 685; Matyjaszewski, K., Ed. Controlled/Living Radical Polymerization. Progress in ATRP, NMP, and RAFT; ACS: Washington, D.C, 2000; ACS Symposium Series 768; Matyjaszewski, K., Davis, T. P., Eds. Handbook of Radical Polymerization; Wiley: Hoboken, 2002; Qiu, J.; Charleux, B.; Matyjaszewski, K. Prog. Polym. Sci. 2001, 26, 2083; and Davis, K. A.; Matyjaszewski, K. Adv. Polym. Sci. 2002, 159, 1.

In several representative embodiments of the present invention, ATRP polymerizations were carried out in an aqueous environment with protein-initiator compositions of the present invention. Similar reactions to the aqueous reactions discussed herein can also be carried out in an organic solvent using techniques described, for example, in U.S. Pat. No. 5,482,996, the disclosure of which is incorporated herein by reference, and other techniques known in the art. In this manner, for example, ATRP of hydrophobic monomers can be initiated. ATRP can, for example, be used in a wide range of solvents to grow polymers from the protein-initiator compositions of the present invention.

Enzymes/proteins can also be pre-modified with an ATRP initiator, ion paired with a surfactant such as sodium bis(2-ethylhexyl) sulfosuccinate (AOT) and dissolved in organic solvents, wherein the protein-initiator composition can be used to grow polymer chains. In this method, an ion pair of a surfactant and the initiator-protein composition are dissolved in the organic solvent and polymerization of, for example, hydrophobic monomers is initiated. Formation of protein-ionic surfactant ion pairs has, for example, been described in Paradkar, V. M and Dordick, J. S. J. Am Chem. Soc. 116, 5009-5010 (1994); Wangikar, P. P., Michels, P. C., Douglas, S. C., Dordick, J. S. J. Am. Chem. Soc. 119, 70-76 (1997) and Novick, S. J., Dordick, J. S., Biomaterials 23, 441-448 (2002), the disclosures of which are incorporated herein by reference. Several representative examples of surfactant and initiator-protein composition ionic pairs and polymerization schemes in organic solvents are described in the attached unpublished manuscript entitled Solvent Soluble Decontaminating Enzymes for Paint Coating, the disclosure of which is incorporated herein by reference and made a part hereof.

A few representative examples of water soluble monomers for use in the present invention include, but are not limited to, mPEG-methacrylate, vinyl pyrrolidone, 2-hydroxypropyl methacrylamide, N-isopropylacrylamide, O-acryloyl-N-acetyl glucosamine, 2-hydroxyethyl methacrylate, dimethylaminoethyl methacrylate, styrene sulfonic acid, N-acryloxysuccinimide, acrylic acid, and/or methacrylic acid. A few representative examples of organic solvent soluble monomers for use in the present invention include, but are not limited to, styrene, methyl methacrylate, butyl methacrylate, 2-ethyl hexyl methacrylate, poly(propylene glycol)methacrylate, perfluoroalkyl methacrylate, vinyl acetate, and/or lauryl acrylate. A few representative examples of ATRP initiators suitable for use in the present invention include, but are not limited to, 2-bromo-2-methyl propionic acid, maleimide derivatives of 2-bromo-2-methyl propionic acid, 2-chloro-2-methyl-propionic acid, and chloro or bromomethyl phenyl carboxylic acids.

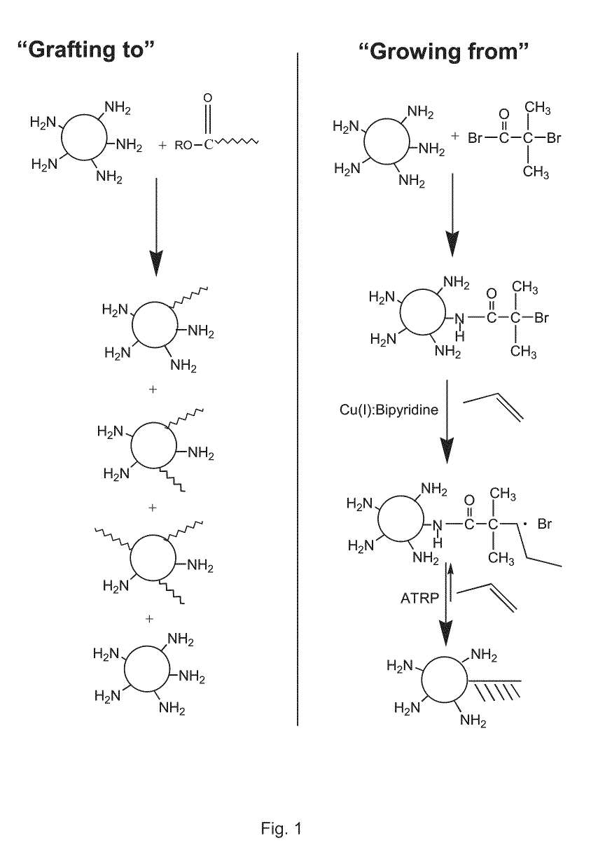

Our approach to growing polymer chains from proteins and the differences between the “grafting-to” and “growing-from” approaches are shown in FIG. 1. Chymotrypsin, a well characterized protein, has been widely used in studies on protein immobilization and modification with polymers. We used chymotrypsin as a model protein in several studies of the present invention. As clear to one skilled in the art, virtually any protein can be conjugated with an initiator as described herein in connection with chymotrypsin using straightforward chemistry. Such proteins can, for example, be used as biocatalysts or pharmaceuticals. As with chymotrypsin, one first modifies the protein with one or more initiators such as ATRP initiators. Representative examples of proteins for use in the present invention include, but are not limited to, subtilisin, lipase, peroxidase, chloroperoxidase, epoxidase, asparaginase, interferon, tumor necrosis factors (TNFs), insulin, superoxide dismutase, and growth factors such as granulocyte macrophage colony stimulating factor (GM-CSF). Once the initators(s) are conjugated with initator, the protein-initiator composition is mixed with at least one monomer which undergoes controlled radical polymerization in the presence of the protein-initiator composition under conditions suitable to initiate the controlled radical polymerization.

To synthesize uniform polymer-protein conjugates by protein-initiated ATRP in the case of chymotrypsin, for example, one first preferably achieves homogenous and controllable modification of, for example, lysine residues with the initiator. Previous studies illustrate that the average number of modified lysine residues in chymotrypsin increased gradually from 2 to 15 as the molar ratio of the acylating agent (pyromellitic anhydride) to chymotrypsin was increased. See, for example, Vinogradov, A. A.; Kudryashova, E. V.; Grinberg, V. Y.; Grinberg, N. V.; Burova, T. V.; Levashov, A. V. Protein Engineering 2001, 14, 683-689.

Synthesis and Characterization of Protein-Initiators

We used 2-bromoisobutyryl bromide as the acylating agent to modify lysine residues in chymotrypsin with 2-bromoisobutyramide groups, the ATRP initiators. In a series of experiments where we measured the degree of modification and enzyme activity, we discovered that the molar ratio of 12:1 for 2-bromoisobutyryl bromide to chymotrypsin gave a single modification resulting in a well-defined protein-initiator that could potentially initiate the ATRP reaction. Further increasing the molar ratio of 2-bromoisobutyryl bromide to chymotrypsin from 43:1 to 85:1 resulted in the conjugation of 3-7 or 7-10 ATRP initiators per protein molecule, respectively. We term these potential protein-initiators as CT¹*, CT⁴* and CT⁸* wherein, the number in the superscript indicates the average number of 2-bromoisobutyramide moieties conjugated to the protein. The yields (in terms of protein recovered) for these “activation” reactions were 80-90% and >90% enzyme activities were retained in all protein-initiators.

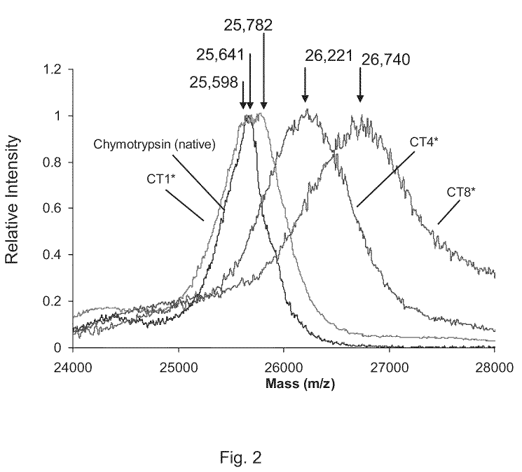

FIG. 2 shows MALDI-TOF spectra of native chymotrypsin, CT¹*, CT⁴* and CT⁸* expanded in the region of mass to charge ratio 24,000 to 28,000. Native chymotrypsin exhibits a molecular ion peak at m/z=25,641. Conjugation of one moiety of 2-bromoisobutyramide will increase molecular weight of chymotrypsin by 150 Da. The molecular ion peak for CT¹* is composed of two peaks merged together (approximately 1:1 mixture of two species) at m/z=25,598 and m/z=25,782. We have assigned these peaks to unmodified chymotrypsin and CT¹*, respectively. We have observed that minor fluctuations are present in m/z values of a protein characterized repeatedly in the same or different MALDI-plate wells. The un-modified chymotrypsin in this preparation cannot initiate polymerization and can therefore be removed from the mixture by dialysis after polymerization. Therefore, we did not attempt to separate the modified and un-modified enzyme. As expected, the molecular ion peaks for CT⁴* and CT⁸* were much broader with peak molecular weights of 26,221 Da and 26,740 Da, respectively.

Growing Polymer Chain from Protein

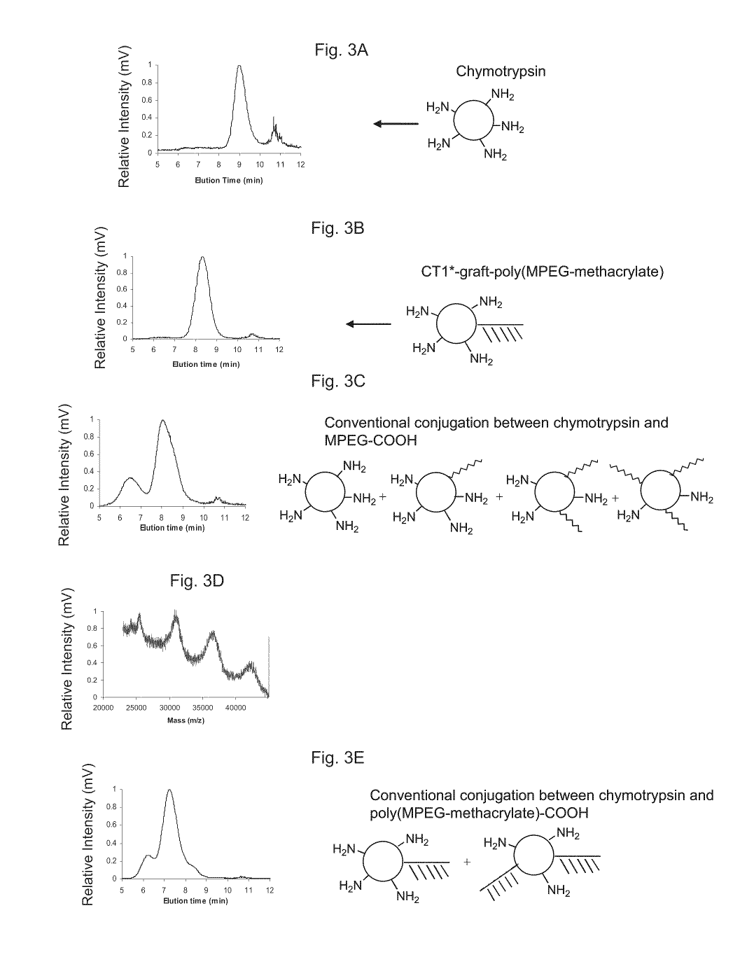

We used MPEG-methacrylate as a macromonomer and initiated its ATRP with CT¹*. The choice of this monomer was based on its efficient polymerization in water at ambient temperatures by ATRP. Wang, X.-S.; Armes, S. P. Macromolecules 2000, 33, 6640-6647. Since there is only one initiator site present on CT¹* it should result in the growth of a single, comb-shaped chain of poly(MPEG-methacrylate) from the protein. We used light scattering detection techniques coupled with size exclusion chromatography (SEC-LS) to determine molecular weights of native and modified proteins. The SEC-LS chromatograms in FIG. 3 tell us that native chymotrypsin elutes as a narrow, Gaussian peak with Mw 26,590 Da (with a small impurity peak with Mw 13,000 Da). CT¹*-graft-poly(MPEG-methacrylate) also exhibits a narrow peak but with Mw 45,600 Da and very low polydispersity index or PDI (1.05).

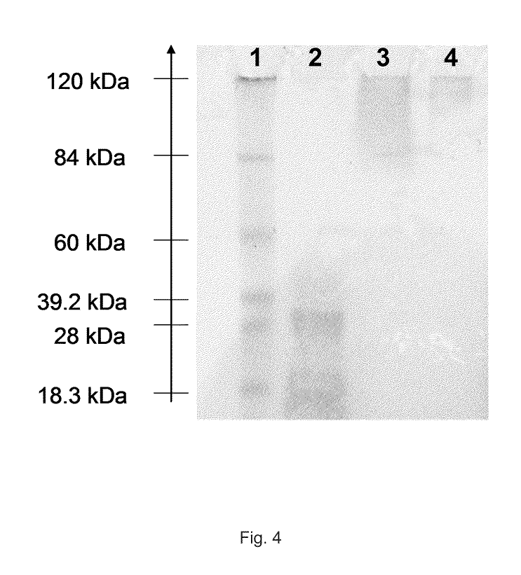

Increased Mw and low PDI both indicate the growth of a near-monodisperse polymer chain from each protein molecule. A PDI of 1, indicate monodispersity. Thus, the protein-polymer conjugates synthesized by our technique are essentially uniform both in terms of the number and the molecular weight of polymer chains attached per protein molecule. Since the Mw of CT¹*-graft-poly(MPEG-methacrylate) is <50,000 Da it is reasonable to assume that there is no formation of a “bridged conjugate” as a result of the addition of polymer chains growing from two different protein molecules. Absence of unmodified chymotrypsin in purified conjugates was confirmed from SDS-PAGE analysis (see FIG. 4). The purification method employed here excludes copper (I) bromide: 2,2′-bipyridine complex by adsorption on silica gel column. The unreacted monomer and/or protein is excluded from the conjugate by dialysis performed using a membrane with MWCO of 30,000 Da. (Mw of chymotrypsin is 25,598 Da).

CT¹*-graft-poly(MEPG-methacrylate) retained 86% of native enzyme activity in the biocatalytic hydrolysis of N-succinyl-L-ala-L-ala-L-pro-L-phe-p-nitroanilide (see Table 1 below). Thus, chymotrypsin can retain substantial activity after the reactive growth of polymer chains from its surface. As the degree of modification and the molecular weight of the conjugates increased from CT⁴* to CT⁸*, the degree of biocatalytic activity retention decreased. This can be attributed to larger and denser polymer chains sterically hindering the access of substrates to the enzyme active site and/or increased deformation of the secondary structure of native protein upon increasing degrees of modification (Table 1). Monomer conversion for CT¹*, CT⁴* and CT⁸* was found to be 17%, 6% and 8%, respectively. These data indicate that as the number of growing polymer chains increase on a protein surface they sterically hinder each other thereby decreasing overall monomer conversion. Proteins modified with one or two initiators can, for example, be used with in the methods of the present invention to achieve both the uniformity in the resulting conjugate as well as a higher percentage of monomer conversion. Recovery of purified protein-polymer conjugates (in terms of the protein collected) was 50-60% in all cases. The relatively low initiation efficiency of protein-initiators seen here is in agreement with the general observation that in ATRP the initiation efficiencies are higher for 2-bromoisobutyrates (ester based initiators) than those for 2-bromoisobutyramides (amide based initiators).

TABLE 1
Molecular weight and activity data for polymer-protein conjugates synthesized
by “growing-from” and “grafting-to” approaches

| | | | Initial Rate | | Initial rate | |
|---|---|---|---|---|---|---|
| | | | at 80 μM | Relative² | at 160 μM | Relative² |
| | | | substrate | activity (%) | substrate | activity (%) |
| Conjugate | Mw¹ (Da) | PDI | (μM/min) | at 80 μM | (μM/min) | at 160 μM |
| Chymotrypsin (control) | 26,700 | 1.05 | 2.95 | 100 | 3.86 | 100 |
| CT¹*-graft-poly(MPEG-methacrylate) | 45,600 | 1.05 | 2.50 | 86 | 3.29 | 85 |
| CT⁴*-graft-poly(MEPG-methacrylate) | 52,350 | 1.17 | 1.60 | 54 | 2.50 | 64 |
| CT⁸*-graft-poly(MEPG-methacrylate) | 90,500 | 1.24 | 1.25 | 42 | 1.81 | 46 |
| Conventional conjugate: | 32,400 | 1.34 | 2.04 | 69 | 3.18 | 82 |
| Chymotrypsin-MPEG 5 kDa | | | | | | |
| Conventional conjugate: | 209,300 | 1.66 | 1.70 | 57 | 2.72 | 70 |
| Chymotrypsin-poly(MPEG- | | | | | | |
| methacrylate) | | | | | | |

¹Molecular weight as determined by SEC-LS chromatography.
²Activities determined using enzyme: substrate ratio 0.2 μM: 80 and 160 μM. Increase in initial rate (of enzyme catalyzed hydrolysis reaction of N-succinyl-L-ala-L-ala-L-pro-L-phe-p-nitroanilide) with increase in the substrate concentration shows that measured rates of hydrolysis are not subjected to substrate saturation of the enzyme.

Comparison of “Growing from” Approach with “Grafting to” Approach

Next, we compared the results of our “growing-from” approach with those of a more traditional “grafting-to” approach. A conjugate was synthesized by combining chymotrypsin and MPEG-SPA in a 1:4 w/w ratio. The SEC-LS chromatogram of this conjugate exhibits a bimodal and broad peak with PDI 1.34 (see FIG. 3(c)). The observed polydispersity is a result of both the multiple sites of modification and the initial polydispersity in MPEG-SPA itself MALDI-TOF spectrometry reveals that this conjugate is a mixture of unmodified and mono-, di- and tri-pegylated enzyme (see FIG. 3(d)). Although the retention time of the major peaks in FIGS. 3(b) and 3(c) are similar, they exhibit light scattering with different intensities, which are used to determine the molecular weights in SEC-LS analysis. Also, high quality MALDI-TOF spectra for chymotrypsins modified with poly(MPEG-methacrylate)s could not be obtained.

In another control experiment, we conjugated chymotrypsin with NHS-activated poly(MPEG-methacrylate) that had been synthesized separately by ATRP (Mw=160,000 Da, PDI=1.30). This conjugate also exhibited a bimodal and broad SEC-LS peak base (FIG. 3(e)). Traditional chemistry does not allow adequate control of the degree of modification. Conjugation reactions using a 1:1 w/w ratio of chymotrypsin and activated polymers resulted in large excess of unmodified enzymes and small amounts of conjugates exhibiting bimodal and broad peaks in SEC-LS chromatograms. As discussed above, the plurality of reactive amino acid residues in a protein limits the uniformity one can achieve in traditional protein modification techniques. For example, Haddleton and coworkers described the synthesis of well-defined, aldehyde-terminated poly(MPEG-methacrylates) and maleimido-terminated poly(MPEG-methacrylates). See Tao, L.; Mantovani, G.; Lecolley, F.; Haddleton, D. M. J. Am. Chem. Soc. 2004, 126, 13220-13221; Mantovani, G.; Lecolley, F.; Tao, L.; Haddleton, D. M.; Clerx, J.; Cornelissen, Velonia, K. J. Am. Chem. Soc. 2005, 127, 2966-2973. Lsozyme or bovine serum albumin was conjugated with these polymers using conventional conjugation techniques. In both the cases SEC analyses of conjugates exhibited broad peaks that are indicative of a mixture of proteins with different degrees of modification.

Possible Sites of Polymer Attachment to Protein

Since the CT¹*-graft-poly(MPEG-methacrylate) appeared to have desirable properties, we studied whether all the protein is modified at the same site, or whether the sample is a mixture of proteins which are all singly modified but at different sites. It is well known that trypsin cuts a peptide following lysine or, to a lesser degree, arginine residues. If one makes a reasonable assumption that polymer-modified lysine residues in modified chymotrypsin will sterically hinder the access of trypsin to that lysine residue, then, any difference that we may observe in size of the peptide fragments obtained from tryptic digests of native and polymer-attached chymotrypsin should provide an insight into the site from which the polymer chain is grown in CT¹*-graft-poly(MPEG-methacrylate).

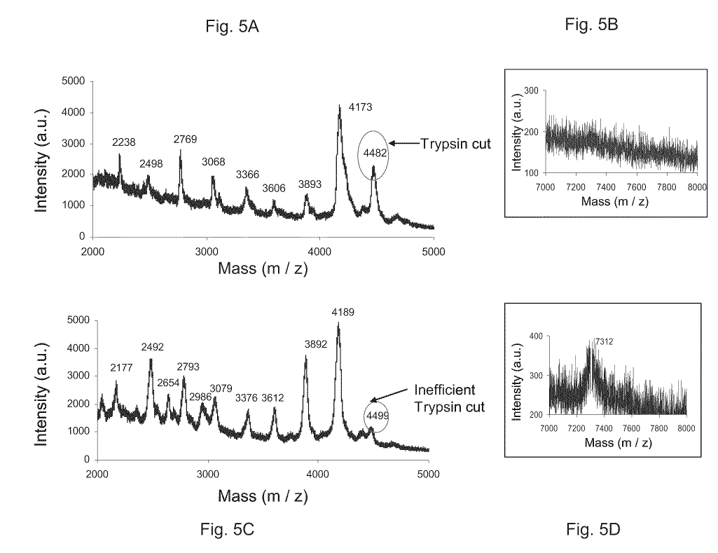

FIGS. 5(a) through 5(d) demonstrate that the relative amount of the 4,500 Da molecular weight fragment is decreased sharply by ATRP and there is simultaneous yet subtle evidence for the emergence of a new fragment having molecular weight 7,300 Da. For the modified protein there is also an unexplainable increase in the peak intensity for peptide fragment of molecular weight 3,382 Da. Since the reactivity of lysine residues in proteins such as RNAase, deoxy-hemoglobin, horseradish peroxidase and sarcosine oxidase have been shown to vary significantly (within same protein) as a result of the differences in their solvent accessibility and pKa values, we are now using computational analyses to predict exactly which lysine is site-specifically growing uniform polymer. Glocker, M. O.; Borchers, C.; Fiedler, W.; Suckau, D.; Przybylski, M. Bioconjugate Chem. 1994, 5, 583-590; Scaloni, A.; Ferranti, P.; De Simone G.; Mamone, G.; Sannolo, N.; Malorni, A. FEBS Lett. 1999, 452, 190-194; O'Brien, A. M., Ó′Fágáin, C., Nielsen, P. F., Welinder, K. G. Biotechnol. Bioeng. 2001, 76, 277-284 and Berberich, J. A.; Lee W. Y.; Madura, J.; Bahar, I.; Russell, A. J. Acta Biomaterialia 2005, 1, 173-181.

Generic Nature of Protein-ATRP Technique

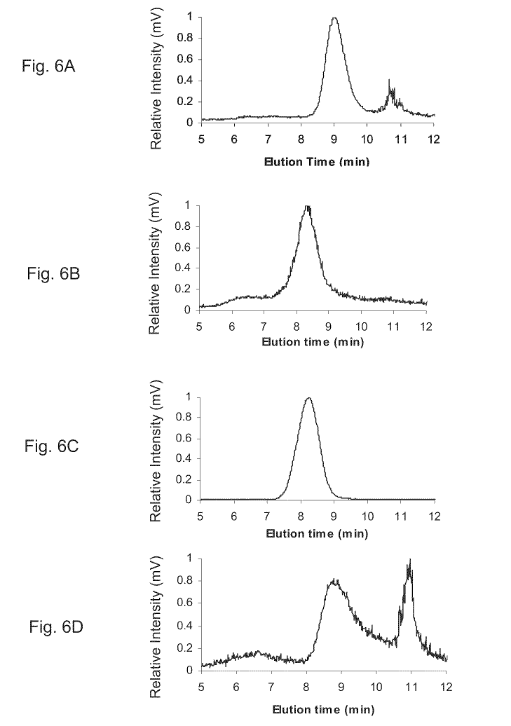

Efficient aqueous ATRP of ionic monomers such as DMAEMA and sodium 4-styrenesulfonate has been reported in the literature. See, for example, Zeng, F.; Shen, Y.; Zhu, S.; Pelton, R. J. Polym. Sci. Part A Polym. Chem. 2000, 38, 3821-3827; Choi, C-K.; Kim, Y-B. Polym. Bull. 2003, 49, 433-439 and Iddon, P. D.; Robinson, K. L. Armes, S. P. Polymer 2004, 45, 759-768. We studied the general applicability of protein-ATRP technique to these monomers. ATRP of DMAEMA (dimethylaminoethyl methacrylate), sodium 4-styrenesulfonate and MPEG-methacrylate (Mw=4.75 Da) was initiated using CT⁴* as a protein-initiator with molar ratio [monomer]/[2-bromoisobutyramide groups]=100. The SEC-LS chromatograms in FIG. 6 show that CT⁴*-graft-poly(sodium 4-styrenesulfonate) and CT⁴*-graft-poly(MPEG475-methacrylate) are obtained with high molecular weights and relatively lower polydispersities. (see Table 2 below). For sodium 4-styerenesulfonate a 63% monomer conversion was obtained. ATRP of DMAEMA initiated from CT⁴* resulted in a very broadly dispersed conjugate along with the formation of poly(DMAEMA) (elution time ˜11 minutes) as a result of the self polymerization of the monomer DMAEMA (FIG. 6(d)). Structurally different protein-polymer grafts retained 40-60% of original enzyme activity (Table 2). Thus, the protein-ATRP technique is capable of producing active and well-defined protein-polymer grafts bearing structurally and functionally different groups.

TABLE 2
Molecular weight and activity data for structurally and functionally
different polymer-chymotrypsin conjugates synthesized by
“growing-from” approach

| | | | Initial rate² | Relative | Initial rate at | Relative |
|---|---|---|---|---|---|---|
| | | | at 80 μM | activity (%) | 160 μM | activity (%) |
| Conjugate | Mw¹ (Da) | PDI | (μM/min) | at 80 μM | (μM/min) | At 160 μM |
| CT⁴*-graft-poly(sodium 4- | 78,150 | 1.10 | 1.36 | 46 | 2.04 | 52 |
| styrenesulfonate) | | | | | | |
| CT⁴*-graft- | 52,200 | 1.08 | 1.81 | 61 | 2.95 | 76 |
| poly(MPEG475- | | | | | | |
| methacrylate) | | | | | | |
| CT⁴*-graft- | 44,300 | 1.35 | 1.13 | 38 | 1.47 | 38 |
| poly(DMAEMA) | | | | | | |

¹Molecular weight as determined by SEC-LS chromatography.
²Activities determined using enzyme: substrate ratio 0.2 μM: 80 and 160 μM. Increase in initial rate (of enzyme catalyzed hydrolysis reaction of N-succinyl-L-ala-L-ala-L-pro-L-phe-p-nitroanilide) with increase in the substrate concentration shows that measured rates of hydrolysis are not subjected to substrate saturation of the enzyme

As described above, we have developed a general method or technique to synthesize active protein-polymer conjugates by conducting a controlled radical polymerization (for example, ATRP) of monomers initiated from protein-displayed initiators. The resulting protein-polymer conjugates are near-monodisperse in terms of the number and molecular weight of polymer chains attached per protein molecule. The technique enables the design and synthesis of well defined and functionally wide ranging polymer-protein conjugates useful, for example, in medicine, catalysis and sensing.

### Experimental Examples

Materials: α-Chymotrypsin (from bovine pancreas), N-succinyl-L-ala-L-ala-L-pro-L-phe-p-nitroanilide, N-hydroxysuccinimide (NHS), sodium phosphate (Na₂HPO₄), bicinchoninic acid solution, copper (II) sulfate solution and bovine serum albumin protein standards were purchased from Sigma Co. (Saint Louis, Mo.). Monomethoxy poly(ethylene glycol)-succinimidyl propionate (MPEG-SPA, molecular weight (Mw)=5,000 Da, polydispersity index (PDI)=1.2) was purchased from Nektar Therapeutics (Huntsville, Ala.). Copper (I) bromide, 2,2′-bipyridine, dichloromethane, n-hexane, dioxane, 1,3-dicyclohexylcarbodiimide (DCC), 2-(dimethylamino)ethyl methacrylate (DMAEMA), sodium 4-styrenesulfonate and MPEG-methacrylate (Mw=1,100 Da and PDI=1.15 (as determined from size exclusion chromatography-light scattering (SEC-LS) technique)) were purchased from Aldrich Chemical Company (Milwaukee, Wis.). Centrifugal dialysis-filtration tubes (Centricon® Plus-20) with 5,000 and 30,000 Da molecular weight cut off (MWCO) were purchased from Millipore Co. (Bedford, Mass.).

Synthesis of protein-initiators (CT¹*, CT⁴* and CT⁸*): Chymotrypsin (1 g, 0.04 mmol protein, (0.52 mmol of —NH₂ groups in lysine residues)) was dissolved in phosphate buffer (100 mL, 0.1 M, pH 8.0). A buffer of pH 8.0 was chosen to increase the nucleophilicity of primary amino groups of lysine residues in the protein towards the acylating reagent. Dichloromethane (5 mL) containing 60, 215 or 425 μL (0.48, 1.74 or 3.43 mmol, respectively) of 2-bromoisobutyryl bromide was slowly added to the enzyme solution and the two-phase reaction mixture was stirred at 800 rpm and at 25° C. Solution pH was maintained at 7.5 to 8.0 by adding 0.1 M sodium hydroxide. Modified proteins were purified by first filtering through a 0.45 μm filter and then by dialysis using Centricon Plus-20 centrifugal dialysis-filtration tubes with MWCO of 5,000 Da. Yields were typically 80 to 90% in terms of the recovered protein. The yields were determined by measuring protein concentration with a bicinchoninic acid assay performed on aliquots of concentrated, purified protein solutions.

Growing polymer chains from protein: Protein-initiator (100 mg, (0.0038, 0.015 or 0.026 mmol of 2-bromoisobutyramide)) was dissolved in phosphate buffer (30 mL, 0.1 M, pH 6.0). The buffer (pH, 6.0) was selected in order to minimize any hydrolysis of the amide bond between chymotrypsin and 2-bromoisobutyramide moiety during the course of polymerization. The appropriate amount of monomer ([MPEG-methacrylate]/[2-bromoisobutyramide groups]=100) was added and the solution was purged with nitrogen gas for 30 minutes at 25° C. The solution was stirred with a magnetic stir bar at 400 rpm. A ten-fold molar excess of the catalyst (1:2 copper (I) bromide: 2,2′-bipyridine complex) over the initiator was added and polymerization allowed to proceed at 40° C. for 16 hrs under nitrogen. Within a few minutes of the catalyst addition, the solution became viscous and dark brown indicating the onset of polymerization. No precipitation was observed over the course of polymerization. Polymer-protein conjugates were purified by passing the dark brown solutions through a silica gel column followed by dialysis using Centricon Plus-20 centrifugal dialysis-filtration tubes with MWCO of 30,000 Da. Yields were typically 50 to 60% in terms of the recovered protein. The yields were determined by measuring protein concentration with a bicinchoninic acid assay performed on aliquots of concentrated, purified protein solutions.

There was no indication of autolysis of chymotrypsin during the course of polymerization (as determined from sodium dodecyl sulfate-polyacrylamide gel electrophoresis (SDS-PAGE).

Synthesis of NHS-activated poly(MEPG-methacrylate): MPEG-methacrylate (Mw=1,100 Da) (4.5 mmol) and N-hydroxysuccinimidyl-2-bromoisobutyrate (0.083 mmol (synthesized by DCC mediated condensation of NHS and 2-bromoisobutyric acid)) were dissolved in a 90:10 water-dioxane mixture. The solution was purged with nitrogen gas for 30 minutes at 25° C. After adding the catalyst (50 mg), polymerization was conducted for 16 hrs at 25° C. under nitrogen, with constant stirring at 400 rpm. The polymer was purified by first passing the dark brown solution through a silica gel column and then treating it with 0.1 M NaOH followed by 0.1M HCl. Finally, the polymer was extracted in dichloromethane and reprecipitated into n-hexane. The re-activation of the carboxyl end group in the polymer was performed via DCC mediated condensation with NHS. Yield 60%.

Synthesis of conventional conjugates: Chymotrypsin (50 mg) and MPEG-SPA (200 mg) were dissolved in 0.1 M phosphate buffer (pH 8.0). The reaction mixture was stirred at 25° C. for one hour and purified using a Centricon Plus-20 dialysis-filtration tube with MWCO of 30,000 Da. Similarly, conjugates with comb polymer were synthesized by reacting chymotrypsin (50 mg) and NHS-activated poly(MPEG-methacrylate) (200 mg). Yields were typically 60 to 70% in terms of the protein recovered in purified conjugates.

Determination of molecular weight: Molecular weights were determined by injecting 100 μL of sample (containing 4 mg/mL protein) into a Waters' gel permeation chromatograph (model Alliance 2695) connected to a Waters' refractive index detector (model 2414) and Precision Detectors' light scattering detector (model PD2020). Samples were passed through Ultrahydrogel Linear column (7.8*300 mm, Waters, catalog No. WAT011545) at the flow rate of 1.0 mL/min using 0.2 M sodium nitrate as the mobile phase. The equations used in the Precision Detectors' Discovery 32 software for molecular weight calculations are as follows.

LS=K_{LS}·C·Mw·(dn/dc)² (1)

RI=K_{RI}·C·(dn/dc) (2)

MW=K′(LS)/(RI) (3)

K′=K_{RI}/[K_{LS}(dn/dc)] (4)

Where, LS is the intensity of the scattered light at 90° angle, K_{LS} is the calibration constant for light scattering detector, C is the concentration of the sample, Mw is the molecular weight of the sample, (dn/dc) is the change in the refractive index of the sample/solvent pair, RI is the refractive index signal, K_{RI} is the calibration constant for refractive index detector and K′ is the instrument calibration constant. Bovine serum albumin (Mw=66,670 Da and dn/dc=0.167 mL/g)) was used as a standard to determine the molecular weights of protein conjugates by LS. We note here that dn/dc of PEG is 0.135 mL/g and it is difficult to determine an exact molecular weight for protein-polymer hybrid systems from measured LS.

Determination of biocatalytic activity: Native chymotrypsin or chymotrypsin-polymer conjugate solution (100 μL, protein concentration 0.1 mg/mL) was added to 1.9 mL of tris(2-aminoethyl)amine hydrochloride (Tris.HCl) buffer (0.1 M, pH 8) in 1 cm path length quartz cell. To this, 100 or 200 μL dimethylsulfoxide containing N-succinyl-L-ala-L-ala-L-pro-L-phe-p-nitroanilide (1 mg/mL) was added and mixed well. The rate of the hydrolysis reaction at 25° C. was monitored for the first 15 seconds by recording increase in the absorbance at 412 nm using a Perkin-Elmer Lambda 45 spectrometer equipped with the software enabled for time dependant absorbance measurement.

Trypsin digestion of protein-polymer conjugates: Native chymotrypsin or chymotrypsin-polymer conjugate (400 mg of protein) and trypsin (20 mg, dimethylated, proteomics grade) were dissolved in 1 mL ammonium bicarbonate buffer (100 mM, pH 8.5). The solutions were incubated at 37° C. for 2 hrs. under stirring.

MALDI-TOF spectrometry of proteins and peptide fragments: A Perseptive Biosystems' Voyager elite MALDI-TOF spectrometer was used to determine the molecular weights of native and modified proteins. The acceleration voltage was set at 20 kV in a linear mode. Protein or peptide solution (0.5-1.0 mg/mL) was mixed with an equal volume of matrix (0.5 mL water, 0.5 mL acetonitrile, 2 μL trifluoroacetic acid and 8 mg 4-hydroxy-3,5-dimethoxy-cinnamic acid) and 2 μL of the resulting mixture was spotted on the plate target. Spectra were recorded after solvent evaporation. Molecular weights of peptide fragments in protein lysates were determined after desalting the solutions using ZipTipC₁₈ microtips (Millipore, cat. No. ZTC1 8S).

The foregoing description and accompanying drawings set forth the preferred embodiments of the invention at the present time. Various modifications, additions and alternative designs will, of course, become apparent to those skilled in the art in light of the foregoing teachings without departing from the scope of the invention. The scope of the invention is indicated by the following claims rather than by the foregoing description. All changes and variations that fall within the meaning and range of equivalency of the claims are to be embraced within their scope.

What is claimed is:

1. A method of synthesizing protein-polymer conjugates; comprising:
reacting a native protein with a reagent, the reagent comprising a group reactive with the reactive sites and a radical polymer initiator, to form a protein-initiator composition retaining biologically activity of the protein in the protein-initiator composition;
mixing the protein-initiator composition with at least one monomer which undergoes controlled radical polymerization in the presence of the protein-initiator composition under conditions suitable to initiate the controlled radical polymerization, wherein the controlled radical polymerization occurs under conditions suitable to maintain biological activity of the protein in the protein-polymer conjugate.

2. The method of claim 1 wherein the controlled radical polymerization is atom transfer radical polymerization.

3. The method of claim 2 wherein the reaction to form the protein-initiator composition is an acylating or alkylating reaction.

4. The method of claim 2 wherein the atom transfer radical polymerization occurs under conditions suitable to maintain a biological activity in the protein-polymer conjugate of at least 10% as compared to a natural biological activity of the protein.

5. The method of claim 2 wherein the atom transfer radical polymerization occurs under conditions suitable to maintain a biological activity in the protein-polymer conjugate of at least 25% as compared to a natural biological activity of the protein.

6. The method of claim 2 wherein the atom transfer radical polymerization occurs under conditions suitable to maintain a biological activity in the protein-polymer conjugate of at least 50% as compared to a natural biological activity of the protein.

7. The method of claim 2 wherein the atom transfer radical polymerization occurs under conditions suitable to maintain a biological activity in the protein-polymer conjugate of at least 75% as compared to a natural biological activity of the protein.

8. The method of claim 2 wherein the atom transfer radical polymerization occurs in an aqueous environment.

9. The method of claim 2 wherein the atom transfer radical polymerization occurs in the presence of a non-aqueous solvent.

10. The method of claim 9 wherein an ion pair of a surfactant and the initiator protein composition is dissolved in the non-aqueous solvent and atom transfer radical polymerization is then initiated.

11. The method of claim 3 wherein the atom transfer radical polymerization occurs at a temperature in the range of 0° C. to 90° C.

12. The method of claim 3 wherein the atom transfer radical polymerization occurs at a temperature of less than 50° C.

13. The method of claim 3 wherein the atom transfer radical polymerization occurs at a temperature of less than 30° C.

14. The method of claim 3 wherein the atom transfer radical polymerization occurs at a temperature of between 0° C. to 25° C.

15. The method of claim 3 wherein the atom transfer radical polymerization occurs under conditions suitable to maintain a biological activity in the protein-polymer conjugate of at least 50% as compared to a natural biological activity of the protein.

16. The method of claim 3 wherein the atom transfer radical polymerization occurs under conditions suitable to maintain a biological activity in the protein-polymer conjugate of at least 75% as compared to a natural biological activity of the protein.

17. The method of claim 2 wherein the ratio of monomer concentration to initiator group concentration is 1 to 100,000.

18. The method of claim 3 wherein the protein is a biocatalyst or a pharmaceutical agent.

19. The method of claim 3 wherein the pH is in the range of approximately 4 to 11.

20. The method of claim 2 wherein a number of initiators covalently attached to the protein is controlled by control of the molar ratio of initiator to protein.

21. The method of claim 20 wherein the molar ratio is controlled to achieve a single initiator covalently attached to the protein.

22. The method of claim 2 wherein the monomer comprises at least one of mPEGmethacrylate, vinyl pyrrolidone, 2-hydroxypropyl methacrylamide, N-isopropylacrylamide, O-acryloyl-N-acetyl glucosamine, 2-hydroxyethyl methacrylate, dimethylaminoethyl methacrylate, styrene sulfonic acid, N-acryloxysuccinimide, acrylic acid, methacrylic acid, styrene, methyl methacrylate, butyl methacrylate, 2-ethyl hexyl methacrylate, poly(propylene glycol)methacrylate, perfluoroalkyl methacrylate, vinyl acetate, or lauryl acrylate.

23. The method of claim 2 wherein the initiator comprises at least one of 2-bromoisobutyryl bromide, 2-bromo-2-methyl propionic acid, a maleimide derivative of 2-bromo-2-methyl propionic acid, 2-chloro-2-methyl-propionic acid, a chloro phenyl carboxylic acid or a bromomethyl phenyl carboxylic acid.

24. The method of claim 2 wherein the protein comprises chymotrypsin, subtilisin, lipase, peroxidase, chloroperoxidase, epoxidase, asparaginase, interferon, a tumor necrosis factor, insulin, superoxide dismutase, or a growth factor.

25. The method of claim 2 wherein the protein is a biocatalyst or a pharmaceutical agent.

26. A composition comprising a protein having covalently attached thereto a controlled radical polymerization initiator.

27. The composition of claim 25 wherein the initiator is an atom transfer radical polymerization initiator.

28. A composition comprising a protein having covalently attached thereto a linking moiety, the linking moiety having covalently attached thereto a polymer chain produced by a controlled radical polymerization, the linking moiety corresponding to an initiator covalently attached to the protein that was used in initiating the controlled radical polymerization.

29. The composition of claim 27 wherein the initiator is an atom transfer radical polymerization initiator.
